(19) United States
(12) Patent Application Publication
(10) Pub. No.: US 2023/0356266 A1
(43) Pub. Date: Nov. 9, 2023

(54) INSTRUMENT CLEANING APPARATUS AND METHOD

(71) Applicant: Innovative Pathology Concepts, Inc., Baltimore, MD (US)

(72) Inventor: Howard Jeffrey Leventhal, Baltimore, MD (US)

(21) Appl. No.: 18/221,664

(22) Filed: Jul. 13, 2023

Related U.S. Application Data
(62) Division of application No. 16/687,596, filed on Nov. 18, 2019.
(60) Provisional application No. 62/788,646, filed on Jan. 4, 2019.

(51) Int. Cl. B08B 3/02; B08B 13/00; B08B 3/14

(52) U.S. Cl. CPC B08B 3/022; B08B 13/00; B08B 3/14

(57) ABSTRACT

Apparatus and method for portable and quick cleaning of an instrument are disclosed. The portable instrument cleaning apparatus may include an instrument inlet to receive a contaminated instrument and a water delivery activation device. The water delivery activation device may comprise a sensor to sense receipt of the contaminated instrument. The water delivery activation device may activate a water delivery system. The water delivery system may convey water from an external water source to the wash tube. The portable instrument cleaning apparatus may pressurize the conveyed water and spray pressurized water on the contaminated instrument in the wash tube. The water delivery activating and instrument cleaning may occur without delay. The sensor may sense removal of the instrument and deactivate the water delivery system. The portable instrument cleaning apparatus may minimize contamination between instrument cleanings using a flushing system and a contaminated water drainage system.

### BACKGROUND OF THE INVENTION

An anatomic pathology laboratory or lab performs diagnostics on a variety of specimens ranging from small biopsies to whole organs and even larger resections of limbs. A diagnostic process begins at the anatomic pathology lab's grossing bench. While a lab may strive to reduce instrument cross-contamination at the grossing bench as much as possible, it happens. In addition to blood, tissue may become attached to dissection instruments and if not properly cleaned, may be transferred from one case to the next. For example, contaminants may comprise cancer cells carried over from previous cases. Accordingly, anatomic pathology labs may experience tissue contamination.

To avoid potential misdiagnosis, lab personnel may devote time away from the diagnostic process to address tissue contamination. They may spend time evaluating whether an artifact on a slide is a contaminant or patient tissue. In addition, they may take on the labor intensive and time-consuming job of keeping their instruments meticulously clean and free of contaminants from other cases. Alternatively, lab personnel may employ a costly approach of using a different set of instruments for each case: instruments may be disposable or collected and cleaned at the end of each day. With this approach, instrument inventory and storage requirements increase. Accordingly, an anatomic pathology lab may balance tissue contamination risks with reduced lab productivity and increased instrument costs.

### SUMMARY OF THE PRESENT INVENTION

Various exemplary embodiments of the present disclosure may demonstrate one or more of the invention features. Other features and advantages of this invention will become apparent from the following detailed description of the presently preferred embodiments of the invention, taken in conjunction with the accompanying drawings.

In accordance with an exemplary embodiment, a portable instrument cleaning apparatus may include an instrument receiving means and a sensor to sense a received instrument. The portable instrument cleaning apparatus may further include a water delivery means and a pressurized water spray system to clean the received instrument with pressurized water. The instrument cleaning apparatus may further include a contaminated water draining system.

In accordance with another exemplary embodiment, a method of portable cleaning of an instrument may include sensing a contaminated instrument. Once a contaminated instrument is sensed, the method of portable cleaning of an instrument may further include activating a water delivery means and spraying pressurized water on the contaminated instrument. The method of portable cleaning an instrument may further include removing contaminated water.

It is to be understood that both the foregoing general description and the following detailed description are exemplary and explanatory only and are not restrictive of the present disclosure or claims.

### BRIEF DESCRIPTION OF THE DRAWINGS

The drawings referenced herein are incorporated in and form part of the specification. The drawings illustrate one or more exemplary embodiments of the present disclosure and together with the description serve to explain various principles and operations. Implications that the drawings illustrate all embodiments of the invention are not to be made.

FIG. 1 illustrates a perspective view of an exemplary embodiment of an instrument cleaning apparatus in accordance with the present disclosure.

FIG. 2 illustrates a perspective view of an exemplary embodiment of a cleaning body of an instrument cleaning apparatus in accordance with the present disclosure.

FIG. 3 illustrates a side cross-sectional view of an exemplary embodiment of an instrument cleaning apparatus in accordance with the present disclosure.

FIG. 4 illustrates a back view of an exemplary embodiment of an instrument cleaning apparatus in accordance with the present disclosure.

FIG. 5 illustrates a bottom view of an exemplary embodiment of an instrument cleaning apparatus in accordance with the present disclosure.

FIG. 6 illustrates a flow diagram showing cleaning an instrument in accordance with the present disclosure.

### DETAILED DESCRIPTION OF THE INVENTION

Reference will now be made in detail to various exemplary embodiments of the present disclosure, examples of which are illustrated in the accompanying drawings.

It will be readily understood that the components of the present invention, as generally described and illustrated in the figures herein, may be arranged and designed in a wide variety of different configurations. Thus, the following detailed description of the embodiments of the instrument cleaning apparatus and method of the present invention, as presented in the figures, is not intended to limit the scope of the invention, as claimed, but is merely representative of selected embodiments of the invention.

Reference throughout this specification to “a select embodiment,” “one embodiment,” or “an embodiment” means that a particular feature, structure, or characteristic described in connection with the embodiment is included in at least one embodiment of the present invention. Thus, appearance of the phrases “a select embodiment,” “in one embodiment,” or “in an embodiment” in various places throughout this specification are not necessarily referring to the same embodiment.

Features, structure, or characteristics described herein may be combined in any suitable manner in one or more embodiments. One skilled in the relevant art will recognize, however, that the invention may be practiced without one or more of the specific details, or with other methods, components, or materials. In other instances, well-known materials or processes are not shown or described in detail to avoid obscuring aspects of the invention. The following description, which shows by way of illustration a specific embodiment in which the invention may be practiced, is intended only by way of example. That is, the following description simply illustrates certain selected embodiments of an instrument cleaning apparatus and method that are consistent with the invention as claimed herein. It is to be understood that other embodiments may be utilized because structural and process changes may be made without departing from the scope of the present invention.

The anatomic pathology lab's grossing bench is the place where the diagnostic process begins. Doctors' offices and surgeries provide samples to the lab, and the lab will decide which pieces to evaluate microscopically. Unlike the operating room, sterile conditions are not necessary; however, under ideal circumstances, the work area should be meticulously clean and all instruments free of contaminants from previous cases. Appropriate attention to ensuring that instruments are not contaminated from previous cases is very labor intensive and time consuming. Unfortunately, the alternative can lead to transfer of material from one case to the next, which may lead to a misdiagnosis and catastrophic results for a patient and a hospital. Indeed, a few cancer cells from one case carried over to the next via the dissecting instruments may result in unnecessary surgery or even far worse scenarios for the patient. Also, for example, in an intrauterine pregnancy there may be millions of chorionic villi, which make up a placenta. In a spontaneous abortion (miscarriage) only one chorionic villus needs to be seen on a microscopic slide to diagnose an intrauterine pregnancy, as opposed to a possible ectopic pregnancy. For an anatomic pathology lab handling multiples of this type of specimen, the chances for contamination from one case to the next may be very high, so a possibility of subsequent misdiagnosis is similarly very high. Accordingly, cross-contamination of grossing bench instruments may affect diagnosis and subsequent patient treatment.

Labs may take actions to prevent or significantly reduce the chances of material being transferred from one case to the next. In one approach, lab personnel may move from the grossing bench to a nearby sink and manually soak or scrub instruments between cases. This approach may be very time consuming and very taxing for most labs and personnel. In another approach, lab personnel may use a different set of instruments for each case. The lab personnel may collect the instruments for cleaning, for example, on a daily basis. This approach may increase the lab's inventory of dissecting instruments, as well as the personnel and facilities to handle the job. In an alternative approach, labs may purchase and use new, disposable instruments for each specimen. The quality of these instruments may not be as good as their non-disposable counterparts, yet the overall cost of disposable instruments may be significantly higher. Further, the lab may maintain a large inventory of disposable instruments, which may increase the lab's working space footprint. Accordingly, reducing chances of cross-contamination between instruments may tax anatomical pathology labs' resources.

An instrument cleaning apparatus and method of the present invention may address these issues. Grossing bench workspace is at a premium. In an exemplary embodiment, the instrument cleaning apparatus may be small and portable. As a result, the instrument cleaning apparatus may be positioned on a working surface, such as the grossing bench, with a limited working surface footprint. In an exemplary embodiment, the instrument cleaning apparatus may comprise a cover, with at least one cover face. In an exemplary embodiment, the cover face may comprise a tool inlet embedded in the cover face. Lab personnel may insert a contaminated instrument into an instrument cleaning apparatus of the present invention via the tool inlet. In an embodiment, for example, a lab personnel motion—the insertion—may occur one-handed. Accordingly, lab personnel may insert contaminated instruments into an instrument cleaning apparatus of the present invention.

Lab personnel may move from a sitting or standing position at a grossing bench to a standing position at a sink to wash contaminated instruments under a faucet. These movements may take time away from performing diagnostic work and may impact lab personnel ergonomically. An instrument cleaning apparatus of the present invention may ergonomically support lab personnel. In an exemplary embodiment, the cover face may be positioned at an angle. As a result, the cover face and the embedded tool inlet may be positioned at the angle. The cover face and the embedded tool inlet may be positioned at an angle that may minimize lab personnel's arm movements when sitting at a grossing bench. In an embodiment, for example, the cover face and embedded tool inlet may be positioned at a 45 degree angle, 60 degree angle, or other angle relative to a surface of the grossing bench. With ergonomic accommodations, in an exemplary embodiment, lab personnel may insert contaminated instruments into an embedded tool inlet with little impact on, for example, lab personnel joints. Additionally, in an embodiment, for example, a position of the cover face and embedded tool inlet relative to lab personnel forearm, hand, elbow, or any combination thereof, may minimize potential contact between lab personnel and contaminated instruments. In addition to issues with contamination, cleaning knives and scalpels can be very dangerous. Accordingly, an instrument cleaning apparatus of the present invention may receive an instrument at an angle.

The cover may encase a cleaning body, and the cleaning body may comprise a wash chamber. In an exemplary embodiment, a wash chamber may comprise a cylindrical shaped body, with a first end, a second end, an inner surface and a cavity. In an exemplary embodiment, the wash chamber may receive a contaminated instrument and pressurized water to wash the contaminated instrument. In an embodiment, for example, the cover may position a tool inlet over the first end of the wash tube. With that in mind, lab personnel may insert a contaminated instrument through the tool inlet, and the cleaning body may receive and position the contaminated instrument in the wash tube cavity. Accordingly, lab personnel may position a contaminated instrument into an instrument cleaning apparatus of the present invention.

In an exemplary embodiment, the cleaning body may comprise a water delivery means. In an embodiment, the water delivery means may comprise, for example, a pump and a valve in communication with the pump. The pump may transport water from a water source through the valve and to a wash tube cavity. In an embodiment, for example, the water source may be an external water source, such as a water faucet or alternative water line. In an alternative embodiment, the external water source may comprise, for example, a self-contained water delivery, such as a jug of water. In an exemplary embodiment, the water delivery means may further comprise a water filter. In this way, an instrument cleaning apparatus of the present invention may minimize adding impurities from the external water source to a received contaminated instrument. Accordingly, an instrument cleaning apparatus of the present invention may comprise a water delivery means.

In an exemplary embodiment, the cleaning body may comprise an activation device. An activation device, for example, may activate an instrument cleaning process. For example, the activation device may activate water or cleaning solution delivery for cleaning an instrument. In an embodiment, lab personnel may activate an instrument cleaning apparatus of the present invention using an “on/off” switch. In an alternative embodiment, an invention of the present disclosure may comprise a hands-free activation device. For example, in an embodiment, lab personnel may activate water or cleaning solution delivery using a foot pedal. In an alternative embodiment, an embodiment of the present invention may comprise an instrument proximity sensor, and the sensor may sense an inserted instrument. For example, the instrument proximity device may detect metals, or other lab instrument or tool materials. In an exemplary embodiment, an invention of the present disclosure may receive a contaminated instrument through a tool inlet, an activation device may sense, for example, the materials of the received instrument, and the activation device may activate water or cleaning solution delivery for cleaning the received instrument. Accordingly, an activation device may activate water or cleaning solution delivery.

In an exemplary embodiment, as described above, an embodiment of the cleaning body may comprise a sensor to identify receipt of a contaminated instrument and initiate a cleaning process. In an exemplary embodiment, the sensor may be positioned to sense when a contaminated instrument crosses a tool inlet or crosses a point within a cavity of the wash tube. Lab personnel want to spend little time waiting for an instrument cleaning apparatus to start cleaning the contaminated instrument. In an exemplary embodiment, an instrument cleaning apparatus of the present invention may provide on-demand instrument cleaning. With that in mind, a sensor may be positioned to sense when a tip of the contaminated instrument enters the wash tube cavity. In an embodiment, for example, a sensor may initiate a cleaning process quickly, substantially immediately when a contaminated instrument crosses a plane within a sensed area. To that end, in an exemplary embodiment, an instrument cleaning apparatus of the present invention may initiate and complete cleaning with little impact on lab personnel work flow. In an exemplary embodiment, a sensor may be embedded in an interior surface of a wash tube. In an alternative embodiment, the sensor may be positioned in the wash tube cavity, extending above the inner surface of the wash tube. Accordingly, a sensor may identify when an instrument cleaning apparatus of the present invention receives a contaminated instrument.

In an embodiment, the sensor may activate a water delivery means. In an alternative embodiment, lab personnel may activate the water delivery means, for example, via a foot pedal. In an exemplary embodiment, activating the water delivery means may comprise opening a valve in communication with a pump. In an exemplary embodiment, opening the value may initiate pumping a flow of water from a water source to a wash tube cavity. Accordingly, an instrument cleaning apparatus of the present invention may transport water from a water source to a wash tube cavity.

In an exemplary embodiment, the cleaning body may comprise a spray system. The spray system may communicate with a water delivery means and receive water from the water delivery means. As described above, lab personnel may insert a contaminated instrument into a tool inlet and position the contaminated instrument in the wash tube cavity. In an exemplary embodiment, the spray system may pressurize water and may spray the pressurized water into the wash tube cavity. As a result, the pressurized water may communicate with the contaminated instrument and may clean the contaminated instrument. In an alternative embodiment, a chemical dispensing system, for example, may communicate with the spray system or the sensor to provide chemical cleaning capabilities in addition to the physical cleaning capabilities. For example, the sensor or the spray system may activate a chemical dispensing system, such as a soap dispensing system, to dispense chemical cleaner into the wash tube cavity. A cleaning body of an instrument cleaning apparatus of the present invention may comprise physical cleaning methods, chemical cleaning methods, or any combination thereof. These methods may reduce, greatly reduce, or possibly eliminate a probability of contamination and subsequent misdiagnosis. In an exemplary embodiment, the chemical dispensing system may dispense soap independent of, or integrated with, the pressurized water. Accordingly, an instrument cleaning apparatus of the present invention may spray pressurized water onto a contaminated instrument.

In an exemplary embodiment, the spray system may comprise at least one water nozzle or water jet. In an embodiment, for example, a water jet may comprise a nozzle, and the nozzle may control water spraying direction and water pressure of water from an external source. In an exemplary embodiment, the at least one water jet may be embedded in the interior surface of a wash tube. In an alternative embodiment, the at least one water jet may be positioned in the wash tube cavity, extending above the inner surface of the wash tube. In an exemplary embodiment, the at least one water jet may have a wide angle spraying range to spray pressurized water on at least a portion of an inserted and received contaminated instrument. In an exemplary embodiment, the spray system may comprise a plurality of water jets. For example, in an embodiment, the spray system may comprise a plurality of water jets, and the water jets may be positioned about approximately equi-distant around the wash tube. Accordingly, an instrument cleaning apparatus of the present invention may use at least one nozzle to spray pressurized water on a contaminated instrument.

In an embodiment, a sensor, as described above, may activate a water delivery means. In an embodiment, the sensor may halt activating the water delivery means. For example, in an embodiment, the sensor may identify material compositions of received instruments. For example, a sensor may identify a metallic material, such as an instrument. For safety reasons, the sensor may identify a “contaminated instrument” as a non-metal material, such as a human finger. In this case, to avoid injury, the sensor may not activate the water delivery means. Accordingly, an instrument cleaning apparatus of the present invention may comprise safety controls.

As described above, a wash chamber may receive a contaminated instrument and pressurized water to wash the contaminated instrument. In an embodiment, the wash chamber may receive the contaminated instrument in a wash tube. In an exemplary embodiment, the wash tube may comprise a first end and a second end. In an exemplary embodiment, a cone-shaped drain basin may communicate and cover the second end. As described above, pressurized water may be sprayed into a wash tube cavity. The pressurized water may clean a contaminated instrument positioned within the wash tube cavity. The water may lose pressure as it communicates with the contaminated instrument, the wash tube cavity, or the inner surface of the wash tube. In an exemplary embodiment, gravity or an applied force may move the previously pressurized, contaminated water down the inner surface and cavity of the wash tube to the drain basin. In an exemplary embodiment, a wash chamber may comprise a drainage tube, and the drain basin may communicate with the drainage tube. In an exemplary embodiment, contaminated water may flow through the drainage tube and exit the cleaning body. Accordingly, an instrument cleaning apparatus of the present invention may drain contaminated water from the apparatus.

As described above, a sensor may identify a contaminated instrument as inserted in the tool inlet or wash tube cavity. In an exemplary embodiment, an instrument cleaning apparatus of the present invention may initiate and complete cleaning quickly, with minimal delay, or seemingly instantaneously after sensing instrument insert. In an embodiment, an instrument cleaning apparatus of the present invention may require little time for cleaning an instrument, so the cleaning may have little impact on lab personnel work flow. Accordingly, an instrument cleaning apparatus may clean an instrument with minimal impact to lab personnel work flow.

Lab personnel may halt the cleaning process by removing an instrument from an instrument cleaning apparatus of the present invention. In an exemplary embodiment, a sensor may identify an instrument as removed from the wash cavity. In an embodiment, the sensor may deactivate the water delivery means. In an exemplary embodiment, the sensor may wait a period of time after sensing a removed instrument before deactivating the water delivery means. For example, the water delivery means may run for some seconds after lab personnel removes an instrument from the wash tube or from the tool inlet. In this way, in an embodiment, a spray system may flush the wash tube cavity of contaminants. Accordingly, an instrument cleaning apparatus of the present invention may flush itself of contaminants.

In an exemplary embodiment, a drainage tube may move contaminated water to an external water capture. For example, in an embodiment, the drainage tube may drain in a sink. In an alternative embodiment, the drainage tube may drain to a self-contained water capture, such as a jug. In an embodiment, a pump may support draining contaminated water from the jug to an external water capture, such as the sink. Accordingly, an instrument cleaning apparatus of the present invention may drain contaminated water externally.

In view of the above described instrument cleaning apparatus and method, the same instruments may be used from one case to the next. Further, instrument cleaning may be quick, almost instantaneous. Apparatus use may be easy and may require only a one-handed operation. Further, device maintenance may be minimal, as self-cleaning may occur with, for example, each use of the instrument cleaning apparatus. Further, a portable instrument cleaning apparatus may require a small footprint, which is critical when space, such as work area space, is at a premium. Accordingly, and instrument cleaning apparatus and method of the present invention may address concerns with contaminated instruments.

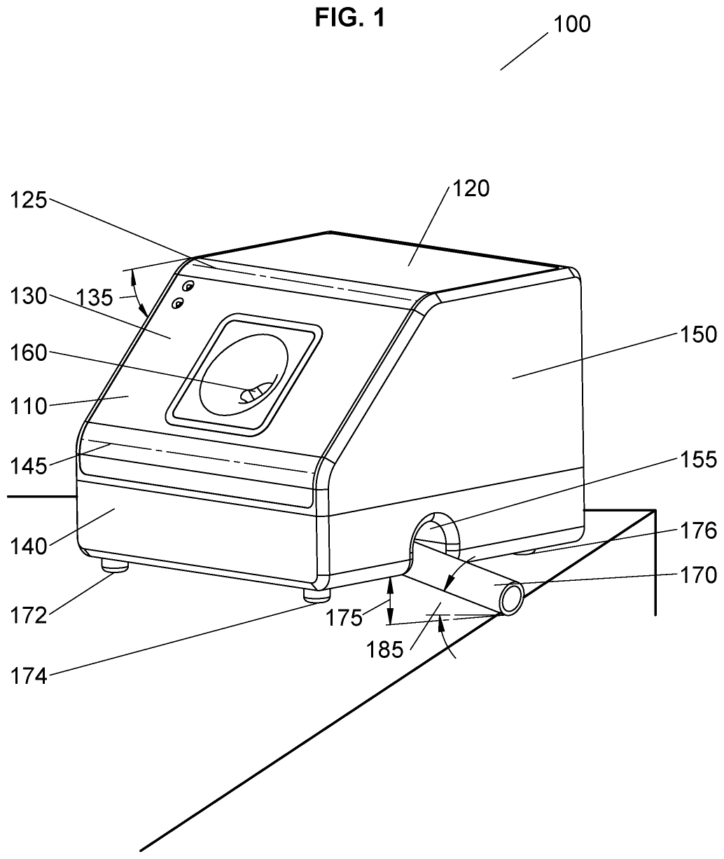

FIG. 1 illustrates a perspective view of an exemplary embodiment of an instrument cleaning apparatus (100) in accordance with the present disclosure. The instrument cleaning apparatus (100) may be used to clean instruments associated with an anatomical pathology laboratory. Similarly, the instrument cleaning apparatus (100) may be used to clean instruments associated with a histology embedding station, a mechanical shop bench, a kitchen counter, or any similar environment. These environments may be similar because a user may work at a substantially horizontal surface and may want to clean at least one instrument while working at the surface. Further, the user may want to clean the at least one instrument on demand, using a small and portable instrument cleaning apparatus (100) as described herein. In an exemplary embodiment, an instrument cleaning apparatus of the present invention may comprise a portable body. Accordingly, an instrument cleaning apparatus of the present invention may clean instruments from a variety of origins.

In an exemplary embodiment, an instrument cleaning apparatus (100) may comprise a cover (110). In an embodiment, for example, the cover (110) may comprise a top face (120) and a bottom face (not shown), opposite and parallel to the top face (120). In an exemplary embodiment, the cover (110) may further comprise a cover face (130), a front apron (140) and a back face (not shown), opposite and parallel to the front apron (140). In an exemplary embodiment, the cover face (140) may communicate with both the top face (120) and the front apron (140). The communication may occur at a first edge (125) and a second edge (145), respectively. In an exemplary embodiment, the cover face (130) may be sloped at an angle (135). As described above, a cover face (130) may be sloped for ergonomic reasons, such that the angle (135) may minimize how high a user may raise an arm to insert an instrument into the instrument cleaning apparatus (100). In an exemplary embodiment, the cover face (130) may further comprise a tool inlet (160). In an exemplary embodiment, the tool inlet (160) may provide an entry point for an instrument into the instrument cleaning apparatus (100). Accordingly, an instrument cleaning apparatus of the present invention may comprise an inlet for instrument entry.

In an exemplary embodiment, the cover (110) may further comprise a left face (not shown) and a right face (150). In an embodiment, for example, the left face (not shown) may comprise a left channel (not shown). In an embodiment, for example, a right face (150) may comprise a right channel (155). In an exemplary embodiment, an instrument cleaning apparatus (100) may comprise the cover (110) and a cleaning body (described below). In an embodiment, the cleaning body (described below) may provide a means for draining contaminated water. In an embodiment, for example, contaminated water may drain from the cleaning body (described below) via a drainage tube (170) positioned within a left channel (not shown) or a right channel (155), or any combination thereof. Accordingly, an instrument cleaning apparatus of the present invention may comprise means for contaminated water egress.

In an exemplary embodiment, an instrument cleaning apparatus (100) of the present invention may comprise support means, such as a first foot (172), a second foot (174), a third foot (176), and a fourth foot (not shown), or any combination thereof. The 4-foot support means is provided for exemplary purposes. In an embodiment, the support means may create a height differential (175) between a bottom face (not shown) and a surface (180). As described above, the surface (180) may be, for example, a grossing bench top, a histology embedding station top, a mechanical shop bench top, a kitchen countertop, or any similar environment work surface. In an exemplary embodiment, the drainage tube (170) may communicate with the surface (180) at an angle (185). The angle may support drainage of contaminated water from the cleaning body (described below). Accordingly, an instrument cleaning apparatus of the present invention may comprise means for supporting contaminated water egress.

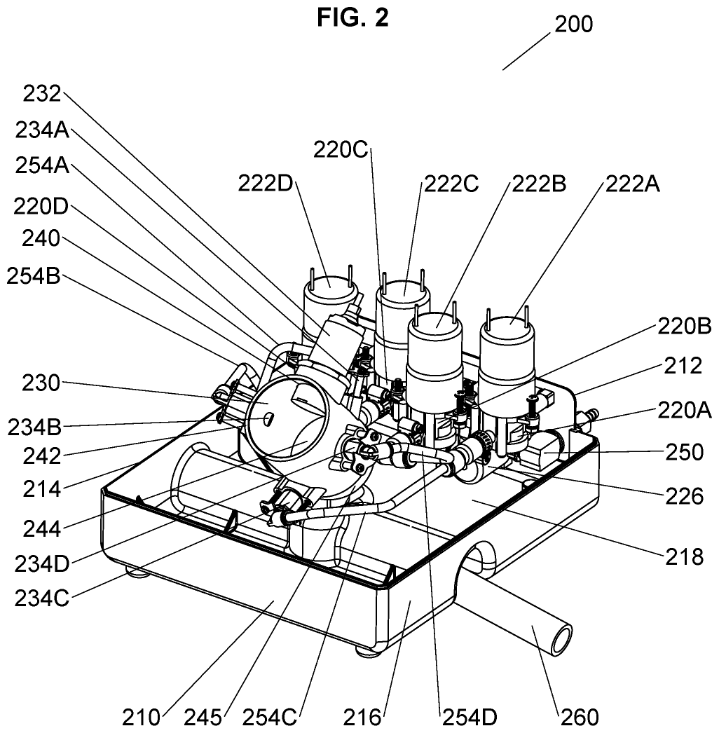

FIG. 2 illustrates a perspective view of an exemplary embodiment of a cleaning body (200) of an instrument cleaning apparatus in accordance with the present disclosure. In an exemplary embodiment, the cleaning body (200) may comprise a front face (210) and a back face (212), opposite and parallel to the front face (210). The cleaning body (200) may further comprise a left face (214) and a right face (216), opposite and parallel to the left face (214). In an exemplary embodiment, the cleaning body (200) may further comprise a floor (218). In an exemplary embodiment, the cover (110) described above may fit over the cleaning body (200) and may attach at the front face (210), back face (212), left face (214), or the right face (216), or any combination thereof. Accordingly, an instrument cleaning apparatus of the present invention may comprise a cover, and the cover may fit over a cleaning body.

In an exemplary embodiment, the cleaning body (200) may further comprise a water delivery means. In an exemplary embodiment, the water delivery means may comprise at least one pump and associated motor. For example, the water delivery means may comprise a first pump (220a), a second pump (220b), a third pump (220c), and a fourth pump (220d). The water delivery means may also comprise, for example, a first motor (222a), a second motor (222b), a third motor (222c), and a fourth motor (222d). The number of pumps and motors is provided for exemplary purposes. In an exemplary embodiment, a water inlet (not shown) may communicate with the back face (212) and at least one pump of the water delivery means to draw water from an external source. In an exemplary embodiment, the at least one pump may be used to increase water pressure between the external source and use in the cleaning body (200) of the present invention. In an embodiment, for example, an energy source, such as a power plug, may communicate with at least one motor of the water delivery means to power at least one pump. Accordingly, an instrument cleaning apparatus of the present invention may access external water and power sources.

In an embodiment, for example, a cleaning body (200) may further comprise a wash chamber for receiving contaminated instruments and pressurized water, and the pressurized water may clean the contaminated instruments. In an embodiment, for example, received pressurized water may flow through at least one pressurized water spraying unit. In an embodiment, the at least one pressurized spraying unit may spray received pressurized water on a received contaminated instrument in the wash chamber. Accordingly, a cleaning body of the present invention may comprise at least one pressurized water spraying units.

In an exemplary embodiment, the cleaning body may comprise a wash tube (230). As described above, the wash tube (230) may communicate with a sensor and a spray system. In an embodiment, for example, the sensor may sense metals, such as metallic instruments used in an anatomic pathology lab. In an exemplary embodiment, a cleaning body (200) may comprise a sensor (232) and a spray system. In an exemplary embodiment, the spray system may comprise a first water jet (234a), a second water jet (234b), and a third water jet (234c), and a fourth water jet (234d), or any combination thereof. The number of water jets is provided for exemplary purposes. In an exemplary embodiment, the surface of each of the first, second, third, and fourth water jets (234a), (234b), (234c), and (234d), may be flush with an inner surface (240) of the wash tube (230). In an alternative embodiment, any combination of the first, second, third, and fourth water jets (234a), (234b), (234c), and (234d), may extend above the inner surface (240) of the wash tube (230). In an exemplary embodiment, the sensor (232) may be flush with the inner surface (240) of the wash tube (230). In an alternative embodiment, the sensor (232) may extend above the inner surface (240) of the wash tube (230). Accordingly, an instrument cleaning apparatus of the present invention may comprise a wash tube with a sensor and at least one water jet.

As described above, in an exemplary embodiment of the present invention, a wash tube (230) may communicate with a sensor (232). In an exemplary embodiment, the sensor (232) may activate the water delivery means, which may comprise at least one pump. That is, the sensor (232) may function as a water delivery activation device. In an alternative embodiment, a hands-free activation device may activate the water delivery means. For example, in an embodiment, a foot pedal may activate the water delivery means. Accordingly, a water delivery activation device may activate the water delivery means to delivery water to a wash chamber.

As described above, a wash tube (230) may comprise a first end (242) and a second end (244). In an exemplary embodiment, a cleaning body (200) may receive a contaminated instrument at the first end (242). In an embodiment, a wash tube (230) may comprise a drain basin (245) at the second end (244). As described above, the drain basin (245) may form a conical-shape, a hemi-spherical shape, or other shape to prevent stasis of water and to support flow of contaminated water from the wash tube (230). In an exemplary embodiment, the drain basin (245) may communicate with a drainage tube (260) for contaminated water egress. Accordingly, an instrument cleaning apparatus of the present invention may comprise a contaminated water drain.

In an exemplary embodiment, a drainage tube (260) may communicate contaminated water through a cleaning device. In an embodiment, for example, the cleaning device may remove contaminates from the contaminated water and supply the cleaned water to the water delivery means. In this way, in an embodiment, water delivery may comprise a closed-loop system. Accordingly, water delivery means of the present invention may communicate with a water cleaning device.

As described above, a water delivery means may comprise at least one pump and at least one motor, and the water delivery means may convey water from an external source to the wash tube (230). In an exemplary embodiment, the at least one pump and the at least one motor may convey water through a valve (226) to a water jet supply system. In an embodiment, a water jet supply system may comprise, for example, a manifold (250) of tubes. In an exemplary embodiment, the manifold (250) may feed water to at least one water jet via at least one tube. For example, in an embodiment, a manifold (250) may feed water to a first water jet (234a) via a first tube (254a), to a second water jet (234b) via a second tube (254b), and to a third water jet (234c) via a third tube (254c). The number of water jets and tubes is provided for exemplary purposes. Accordingly, an instrument cleaning apparatus may supply water to at least one water jet for cleaning a contaminated instrument.

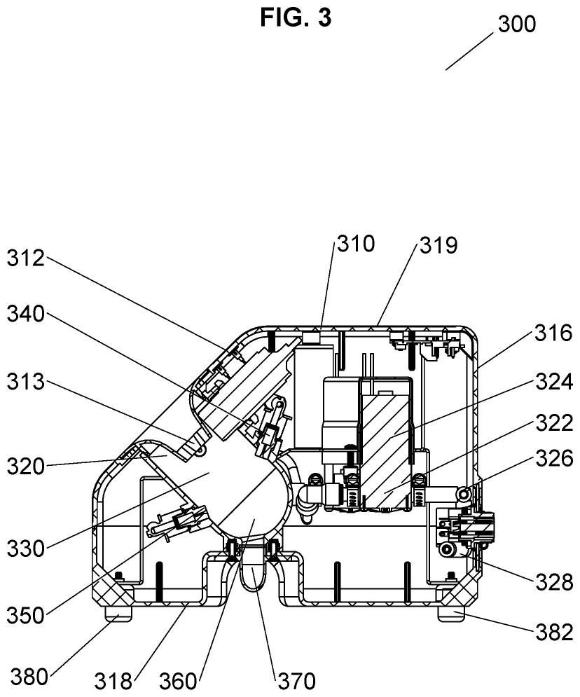

FIG. 3 illustrates a side cross-sectional view of an exemplary embodiment of an instrument cleaning apparatus (300) in accordance with the present disclosure. In an exemplary embodiment, an instrument cleaning apparatus (300) may comprise a cover (310). In an exemplary embodiment, the cover (310) may comprise a cover face (312), and the cover face (312) may comprise a tool inlet (313). In an exemplary embodiment, the cover (310) may comprise a front apron (314) and a back face (316), which may be opposite and parallel to the front apron (314). In an exemplary embodiment, the cover (310) may further comprise a bottom face (318) and a top face (319), which may be opposite and parallel to the bottom face (318). Accordingly, an instrument cleaning apparatus may comprise a cover.

In an exemplary embodiment, the instrument cleaning apparatus (300) may comprise a wash chamber (320). As described above, in an exemplary embodiment, the cleaning body may comprise a water delivery means. In an embodiment, for example, the water delivery means may comprise a pump (322), motor (324), and a manifold (326) with at least one tube (328). In an exemplary embodiment, the at least one tube (328) may provide water to at least one water jet (340). In an exemplary embodiment, a wash chamber (320) may comprise a wash tube (330) in communication with the at least one water jet (340) and a nozzle (350). In an exemplary embodiment, a wash chamber (320) may comprise a plurality if wash tubes. In an exemplary embodiment, the wash tube (330) may communicate with a drain basin (360), and the drain basin (360) may communicate with a drainage tube connection (370). Accordingly, an instrument cleaning apparatus may comprise a cover and a wash chamber.

As described above, an instrument cleaning apparatus of the present invention may encourage contaminated water drainage using, for example, gravity. Specifically, the bottom face (318) of the instrument cleaning apparatus (300) may be positioned a height above a flat surface, such as a working surface. As a result, a drainage tube connection (370) may be positioned higher at a point in communication with the instrument cleaning apparatus (300) relative to a point in communication with the working surface. In an embodiment, a drainage tube connection (370) may comprise, for example, a drainage tube or, for example, a drainage tube outlet assembly, such as a drainage tube and a connector piece. In an embodiment, for example, an instrument cleaning apparatus (300) may further comprise support means, such as a first foot (380) and a second foot (382). The number of support structures is provided for illustration and exemplary purposes. Accordingly, an instrument cleaning apparatus may support contaminated water drainage with height supports.

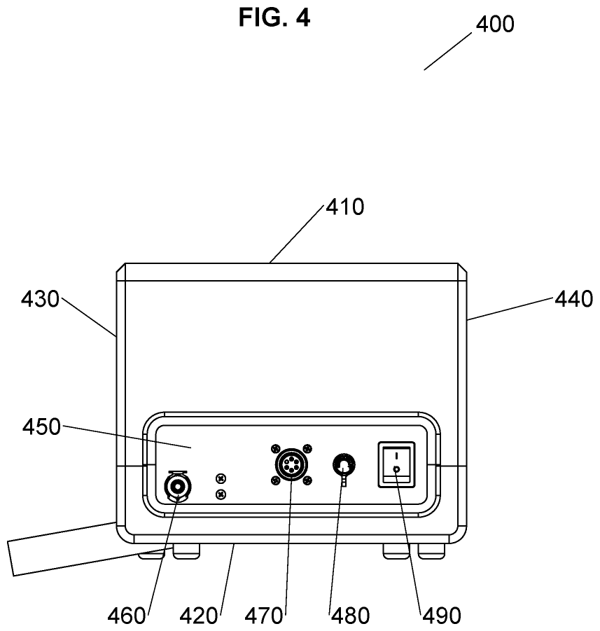

FIG. 4 illustrates a back view (400) of an exemplary embodiment of an instrument cleaning apparatus in accordance with the present disclosure. In an exemplary embodiment, a back view (400) may comprise a top edge (410) opposite and parallel to a bottom edge (420). The back view (400) may further comprise a left edge (430) opposite and parallel to a right edge (440). The back view (400) may further comprise panel (450). In an embodiment, the panel (450) may comprise, for example, a water inlet (460) and a power inlet (470). In an embodiment, the panel (450) may further comprise, for example, a foot switch jack (480), which may be used for foot pedal switch operations. For example, in an embodiment, rather than a sensor identifying a received contaminated instrument in wash chamber and activating a water delivery system, a hands-free device, such as a foot pedal, may activate the water delivery system. In another exemplary embodiment, the panel may further comprise a power switch (490). Accordingly, an instrument cleaning apparatus of the present invention may comprise water and power inlets.

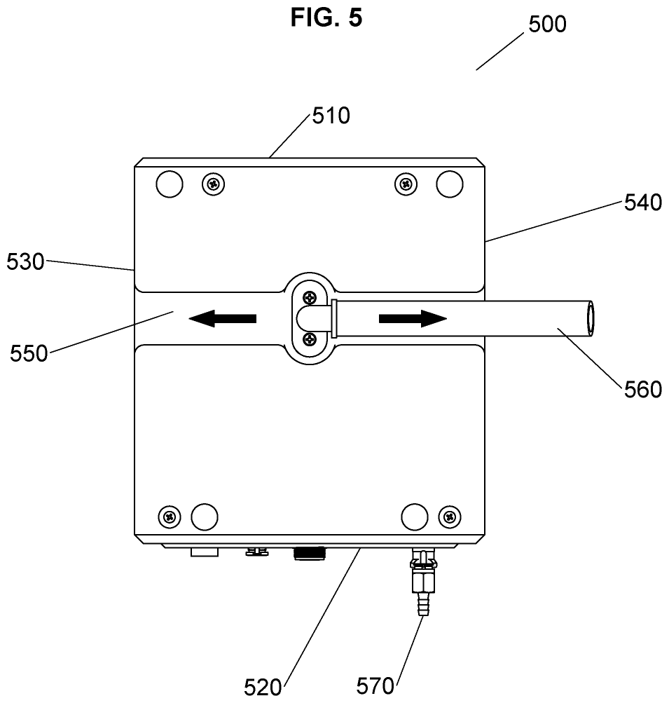

FIG. 5 illustrates a bottom view (500) of an exemplary embodiment of an instrument cleaning apparatus in accordance with the present disclosure. In an exemplary embodiment, a bottom view (500) may comprise a front edge (510) opposite and parallel to a back edge (520), and a left edge (530) opposite and parallel to a right edge (540). In an embodiment, for example, a bottom view (500) may further comprise a drain tube channel (550). In an exemplary embodiment, the drain tube channel (550) may extend from the left edge (530) to the right edge (540), across the bottom view (500). As described above, a drain basin may communicate with a drainage tube at a drain. In an exemplary embodiment, a drainage tube (560) may fit within a drain tube channel (550), so that water may egress from the instrument cleaning apparatus. In an embodiment, the drainage tube (560) may communicate with half of the drain tube channel (550), such that water may drain past the left edge (530) or the right edge (540). In an embodiment, for example, a bottom view (500) of an instrument cleaning apparatus may comprise a water inlet (570). Accordingly, an instrument cleaning apparatus of the present invention may drain water from the instrument cleaning apparatus.

In an embodiment of the present invention, use of a portable, on-demand, instrument cleaning apparatus may comprise receiving an instrument in the portable apparatus and activating a water delivery system. Use of the portable apparatus may further comprise delivering water to a water spray system and spraying the delivered water on the received, contaminated instrument. In an embodiment, for example, use of the portable apparatus may further comprise deactivating the water delivery system. Accordingly, an embodiment of the present invention may comprise spraying water on an instrument received in a portable, on-demand, instrument cleaning apparatus.

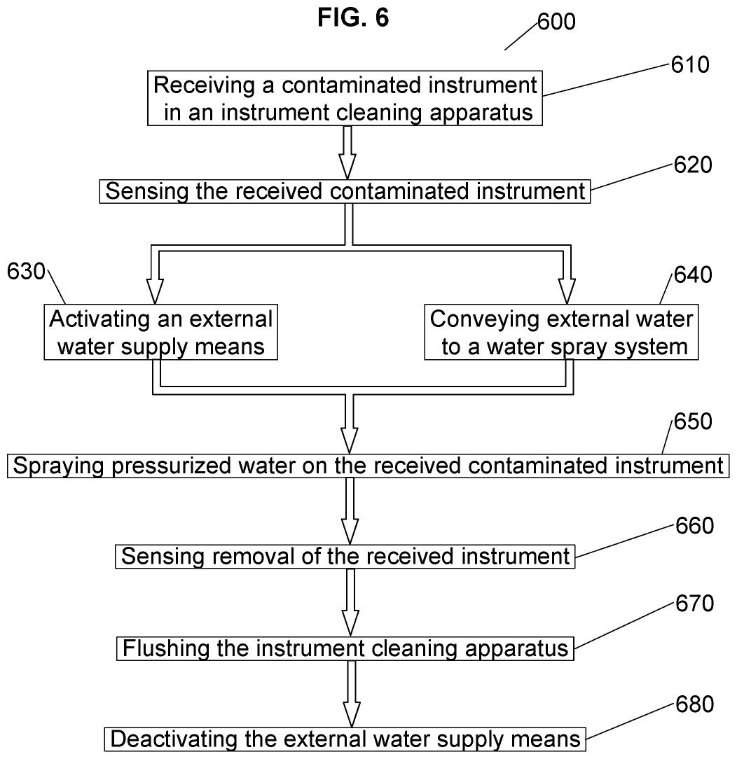

FIG. 6 illustrates a flow diagram showing cleaning an instrument (600) in accordance with the present disclosure. In an exemplary embodiment, cleaning an instrument (600) may comprise receiving a contaminated instrument in an instrument cleaning apparatus (610), sensing the received contaminated instrument (620), and activating an external water delivery means (630). In an embodiment, sensing the received contaminated instrument (620), and activating an external water delivery means (630), together, may comprise auto-sensing, particularly, for example, when metallic instruments enter a tool inlet (160), as described in FIGS. 1-3. As described above, in an alternative embodiment, cleaning an instrument (600) may comprise receiving a contaminated instrument 610) and activating an external water delivery means (630) with a hands-free device, such as a foot pedal. In an exemplary embodiment, a foot pedal may be used, for example, when non-metallic instruments enter a tool inlet (160), as described in FIG. 1. In an embodiment, for example, a foot pedal may allow for hands-free use of the present invention. In an exemplary embodiment, cleaning an instrument (600) may further comprise conveying external water to a water spray system (640) and spraying pressurized water on the received contaminated instrument (650). In an embodiment, for example, conveying external water to a water spray system (640) may comprise opening valves and activating water supply means, such as pumps and motors, as described in FIGS. 2 and 3. In an exemplary embodiment, cleaning an instrument (600) may further comprise sensing removal of the received instrument (660), flushing the instrument cleaning apparatus (670), and deactivating the external water delivery means (680). In an embodiment, for example, cleaning an instrument (600) may also comprise continuously draining contaminated water from the instrument cleaning apparatus of the present invention. Accordingly, cleaning an instrument may comprise receiving a contaminated instrument in an instrument cleaning apparatus, spraying pressurized water on the received contaminated instrument, and draining the contaminated water from the instrument cleaning apparatus.

It is to be understood that the various embodiments shown and described herein are to be taken as exemplary. Elements and materials, and arrangements of those elements and materials, may be substituted for those illustrated and described herein, parts may be reversed, and certain features of the present disclosure may be utilized independently, as would be apparent to one skilled in the art after having the benefit of the description herein. Changes may be made in the elements described herein without departing from the spirit and scope of the present disclosure and following claims, including their equivalents.

It is to be understood that the particular embodiments set forth herein are non-limiting, and modifications to structure, dimensions, materials, and methodologies may be made without departing from the scope of the present disclosure.

It is to be further understood that this description's terminology is not intended to limit the invention. For example, spatially relative terms, such as “front,” “back,” “top,” “bottom,” “side,” and the like, may be used to describe one element's or feature's relationship to another element or feature as intended to connote the orientation of, for example, the instrument cleaning apparatus as illustrated in the figures.

For the purposes of this specification and appended claims, unless otherwise indicated, all numbers expressing quantities, percentages or proportions, and other numerical values used in the specification and claims, are to be understood as being modified in all instance by the term “about” if they are not already. That is, unless indicated to the contrary, the numerical parameters set forth in the specification and claims are approximations that may vary depending on the desired properties sought to be obtained by the present disclosure.

1. A method, the method comprising:
receiving an instrument through a first end of a funnel-shaped instrument inlet positioned 45-60 degrees from horizontal in a portable apparatus;
receiving the instrument in a wash tube in linear alignment with the funnel-shaped instrument inlet through a second end of the funnel-shaped instrument inlet, wherein the first end of the funnel-shaped instrument inlet is larger than the second end of the funnel-shaped instrument inlet;
sensing the received instrument material composition;
activating a water delivery system;
spraying delivered water on the received instrument;
stopping overspray of the sprayed delivered water against the first end of the funnel-shaped instrument inlet;
capturing the sprayed delivered water in a contaminated water drain basin positioned in linear alignment with the funnel-shaped instrument inlet and wash tube; and
deactivating the water delivery system.

2. (canceled)

3. The method of claim 7, wherein deactivating the water delivery system comprises sensing removal of the received instrument.

4. The method of claim 7, further comprising pressurizing the delivered water.

5. The method of claim 7, further comprising flushing the portable apparatus with delivered water.

6. The method of claim 7, further comprising continuously draining sprayed water.

7. The method of claim 7, wherein activating a water delivery system comprises depressing a foot pedal.
